(19) United States
(12) Patent Application Publication
(10) Pub. No.: US 2008/0287981 A1
(43) Pub. Date: Nov. 20, 2008

(54) DILATION INTRODUCER AND METHODS FOR ORTHOPEDIC SURGERY

(75) Inventors: Brad S. Culbert, Rancho Santa Margarita, CA (US); Mark C. Boomer, Phoenix, AZ (US); Christopher Warren, Aliso Viejo, CA (US)

Correspondence Address: KNOBBE MARTENS OLSON & BEAR LLP, 2040 MAIN STREET, FOURTEENTH FLOOR, IRVINE, CA, 92614, US

(73) Assignee: Interventional Spine, Inc., Irvine, CA (US)

(21) Appl. No.: 12/124,966

(22) Filed: May 21, 2008

Related U.S. Application Data
(62) Division of application No. 10/911,215, filed on Aug. 3, 2004.

(51) Int. Cl. A61B 19/00; A61M 19/00; A61M 5/178

(52) U.S. Cl. 606/191; 604/164.13; 128/898

(57) ABSTRACT

A method is provided for a minimally invasive procedure utilizing the telescoping dilation introducer to insert a bone fixation device into a patient's spine for posterior spine fusion. The dilation introducer has a locked assembled configuration for placement of the dilation introducer against a patient's bone tissue to be treated, and an unlocked, collapsed configuration for dilating the patient's soft tissue down to the bone tissue to be treated to a desired degree of dilation to permit minimally invasive surgical procedures on the patient's bone tissue to be treated. Dilator tubes are successively released and advanced to progressively expand the patient's soft tissue down to the bone tissue to be treated.

### CROSS-REFERENCE TO RELATED APPLICATIONS

This application is a divisional of U.S. application Ser. No. 10/911,215, filed on Aug. 3, 2004, the entire contents of which is hereby incorporated herein by reference.

### BACKGROUND OF THE INVENTION

1. Field of the Invention

This invention relates to bone fixation devices, and more particularly relates to a dilation introducer for introducing a bone fixation device for orthopedic surgery, such as for vertebral fusion.

2. Description of the Related Art

Fusion of two adjacent vertebrae is a common surgical treatment for back injuries due to damage or defects in a spinal disc between two adjacent vertebrae, such as conditions due to a herniated disc or disc degeneration. The entire disc may be removed by a discectomy procedure, and may be replaced with bone or a bone substitute and/or cage in order to prevent collapse of the disc space between the adjacent vertebrae. Early techniques for stabilizing the adjacent vertebrae included application of a plate or a rod in conjunction with screws across the adjacent vertebrae, after which the adjacent vertebrae would eventually fuse together. However, such techniques commonly required prolonged periods of recovery from the extensive surgery involved, and it would be desirable to provide an improved apparatus and method for providing a minimally invasive procedure that will result in less trauma and improvement in patient recovery.

Bone fixation devices are known that are useful for connecting two or more bone segments for the healing of broken bones, typically including an elongate pin with a distal anchor and a proximal anchor movable on the pin to accommodate different bone dimensions, and to permit tensioning of the bone segments together. The surgical procedure of attaching two or more parts of a bone with a pin-like device commonly requires an initial incision into the tissue down to the bone, and the drilling of a hole through the bone parts to be joined. Such bone fixation devices can be useful for fusion of vertebrae together, because such a bone fixation device can be used to join adjacent bone segments through a single percutaneous incision or puncture, without the need to expose any other side of the bone segments to be joined. In either type of procedure, there is substantial trauma to the surrounding tissue if a large incision is required. Thus, it would be desirable to provide a minimally invasive dilation introducer to allow the penetration and spreading of soft tissues down to vertebrae to be fused, for use of such a bone fixation device to join adjacent vertebrae, and to allow for more easily performing the delicate maneuvering of drilling adjacent vertebrae and application of one or more bone fixation devices to join the vertebrae to be fused. The present invention satisfies these and other needs.

### SUMMARY OF THE INVENTION

Briefly, and in general terms, the invention provides for a telescoping dilation introducer for orthopedic surgery, the dilation introducer having a locked assembled configuration for initial placement of the dilation introducer against a patient's tissue to be treated, and an unlocked, collapsed configuration for dilating the patient's soft surrounding tissue to a desired degree of dilation to permit minimally invasive surgical procedures on the patient's tissue. As the telescoping dilation introducer is inserted, each individual dilator tube is successively released and advanced to progressively expand the patient's soft tissue down to the tissue to be treated. In a particularly useful aspect of the invention, the tissue to be treated is bone tissue which must be prepared prior to attachment of adjacent bone section in a fusion process. While there are many applications of the dilation introducer of the invention, the invention is particularly applicable to fusion of bones in orthopedic surgery using minimally invasive technique, and will be described herein in particular applications of those procedures. The invention also concerns a minimally invasive procedure utilizing the telescoping dilation introducer to insert a bone fixation device into a patient's spine for posterior spine fusion. While posterior spine fusion currently takes up to two hours to complete, and requires a six inch incision, with the apparatus and method of the invention, comparable surgery can be completed in less than thirty minutes, with a dilation port 13 mm or less in diameter, thus lowering the chance of damage to the surrounding soft tissue.

The present invention accordingly provides for a dilation introducer for orthopedic surgery, the dilation introducer having a locked assembled configuration for initial placement of the dilation introducer against a patient's bone tissue to be treated, and an unlocked, collapsed configuration for dilating the patient's soft tissue to a desired degree of dilation to permit minimally invasive surgical procedures on the patient's bone tissue. The dilation introducer includes a first dilator tube having a distal end and a proximal end, a longitudinal lumen with a distal opening and a proximal opening, the distal end having a tapered tip, and a second dilator tube, the first dilator tube being removably received in the second dilator tube for slidable telescoping movement within the second dilator tube, the second dilator tube having a distal end and a proximal end, an inner lumen with a distal opening and a proximal opening, the distal end having a tapered tip. Alternatively, the first dilator tube may have a non-cannulated configuration, formed without a lumen. The dilation introducer also advantageously includes means for removably connecting the first and second dilator tubes together in a locked configuration, whereby in the locked configuration the distal end of the first dilator tube can be pressed against the patient's bone tissue to be treated, and whereby in the unlocked configuration the second dilator tube is permitted to slidably telescope over the first dilator tube to dilate the patient's soft tissue at the distal end of the dilation introducer.

While the invention will be described with specificity to a spinal fusion procedure, those skilled in the art will recognize that the apparatus and method of the art will recognize that the apparatus and method of the invention can also be advantageously used for procedures in which the dilation introducer can be brought up against other firm or solid structures in the body, or placed in the body, to thereby gain the advantages of the invention for other minimally invasive procedures.

In a presently preferred aspect, the dilation introducer also includes one or more additional distally tapered dilator tubes, and the second dilator tube is removably received within the one or more additional dilator tubes, in a locked configuration in which the distal end of the second dilator tube can be pressed against the patient's bone tissue to be treated, and in an unlocked configuration in which the one or more dilator tubes can slidably telescope over the second dilator tube to dilate the patient's soft tissue at the distal end of the dilation introducer. In another presently preferred aspect, the distal ends of the first dilator tube, the second dilator tube, and the one or more additional dilator tubes may have a tapered, beveled tip. In another aspect, at least one plastic sleeve may be slidably disposed over the at least one additional dilator tube, and the distal end of the plastic sleeve may also have a tapered, beveled tip.

In a first embodiment, the proximal end of the first dilator tube includes a pair of spaced apart rings, the proximal end of the second dilator tube includes a pair of spaced apart rings, and the means for removably connecting the first and second dilator tubes together in a locked configuration comprises a first locking clip removably connected to the first dilator tube between the pair of spaced apart rings of the first dilator tube and to the second dilator tube between the pair of spaced apart rings of the second dilator tube, and wherein removing the first locking clip allows the second dilator tube to slidably telescope over the first dilator tube to further dilate tissue at the distal end of the dilation introducer.

In a preferred aspect, the first locking clip includes a first portion and a second portion, and a cross-piece having a first end and a second end connected between the first portion and the second portion, the first portion including a pair of resilient arms each having a proximal narrow neck portion connected to the cross-piece and a distal gripping portion extending from the narrow neck portion, the pair of resilient arms having an inner rounded surface adapted to snap over the first dilator tube between the spaced apart rings of the first dilator tube, the second portion including a single arm having a proximal narrow neck portion connected to the cross-piece and a distal gripping portion extending from the narrow neck portion, the single arm having an inner rounded surface adapted to fit over the outer surface of the second dilator tube between the spaced apart rings of the second dilator tube, to connect the first and second dilator tubes.

In another aspect, the proximal end of the second dilator tube includes a pair of spaced apart rings, the proximal end of the at least one additional dilator tube includes a handle fixedly mounted to the proximal end of the at least one additional dilator tube, and the means for removably connecting the second dilator tube and the at least one additional dilator tube together in a locked configuration includes a second locking clip removably connected to the first dilator tube between the pair of spaced apart rings of the second dilator tube and to the at least one additional dilator tube over the handle, and wherein removing the second locking clip allows the at least one additional dilator tube to slidably telescope over the second dilator tube to further dilate tissue at the distal end of the dilation introducer.

In a preferred aspect, the second locking clip comprises a first portion and a second portion, and a cross-piece having a first end and a second end connected between the first portion and the second portion, the first portion including a pair of resilient arms each having a proximal narrow neck portion connected to the cross-piece and a distal gripping portion extending from the narrow neck portion, the pair of resilient arms having an inner rounded surface adapted to snap over the second dilator tube between the spaced apart rings of the first dilator tube, the second portion including a pair of resilient arms each having a proximal narrow neck portion connected to the cross-piece and a distal gripping portion extending from the narrow neck portion, the pair of resilient arms having an inner rounded surface adapted to snap over the at least one additional dilator tube to connect the second dilator tube and the at least one additional dilator tube.

In a second embodiment, the means for removably connecting the first and second dilator tubes together in a locked configuration comprises a latching member projecting from the proximal end of the first dilator tube toward the distal end of the first dilator tube, and the proximal end of the second dilator tube includes a head with a first radial aperture removably receiving a first locking pin, and a second longitudinal aperture for removably receiving the latching member, the latching member being engaged by the first locking pin, and whereby removing the first locking pin from the first radial aperture releases the latching member to permit the second dilator tube to slidably telescope over the first dilator tube to further dilate tissue at the distal end of the dilation introducer.

In a preferred aspect of the second embodiment, the means for removably connecting the second dilator tube and the at least one additional dilator tube together in a locked configuration comprises a latching member projecting from the proximal end of the second dilator tube toward the distal end of the second dilator tube, and the proximal end of the at least one additional dilator tube includes a first radial aperture removably receiving a second locking pin, and a second longitudinal aperture for removably receiving the latching member, the latching member being engaged by the second locking pin, and whereby removing the second locking pin from the first radial aperture releases the latching member to permit the at least one additional dilator tube to slidably telescope over the second dilator tube to further dilate tissue at the distal end of the dilation introducer.

In another aspect, the means for removably connecting the first and second dilator tubes together in a locked configuration comprises a bayonet fitting removably coupling the first and second dilator tubes together. In a third embodiment, the bayonet fitting comprises a first pair of opposing bayonet pins extending from the proximal end of the first dilator tube, and interior opposing bayonet slots formed in the second dilator tube for receiving the first pair of opposing bayonet pins of the first dilator tube. In a preferred aspect of the third embodiment, the means for removably connecting the second dilator tube and the at least one additional dilator tube together in a locked configuration comprises a bayonet fitting removably coupling the second dilator tube and the at least one additional dilator tube together, and the bayonet fitting may include a pair of opposing bayonet pins extending from the proximal end of the second dilator tube, and interior opposing bayonet slots formed in the at least one additional dilator tube for receiving the pair of opposing bayonet pins of the second dilator tube. In a fourth embodiment, the bayonet fitting comprises a bayonet pin extending from the proximal end of the first dilator tube, and a bayonet slot formed in the proximal end of the second dilator tube for receiving the bayonet pin of the first dilator tube.

In another presently preferred aspect, the dilation introducer may further include a tubular bone drill removably received in the first or subsequent dilator tube; and a guide wire may also be removably received in the tubular bone drill to contact the bone tissue to be treated.

In another aspect, the invention provides both a means of locating the various elements of the invention by fluoroscopy when the elements are not made of radiopaque markers, such locating means including radiopaque bands or portions of the elements located in predetermined places on the dilator elements to allow for visualization of their use in the body by fluoroscopy or the like.

The present invention also provides for a method of dilating a patient's soft tissue down to the bone tissue to be treated in orthopedic surgery. An entry point is located on the bone tissue to be treated, and the tip of a guide wire is placed at the entry point on the bone tissue to be treated and advanced to the soft tissue of the patient to the target point of the inferior articular facet. A vertical midline incision to a desired depth is made in the skin and fascia of the patient, using the entry point as the middle of the incision. A first dilator tube of the dilation introducer is then passed over the guide wire until the tip of the dilation introducer reaches the target point of the bone. The guide wire is then driven into the facet joint and into the pedicle of the patient, with verification of the trajectory and depth by fluoroscopy. The second dilator tube of the dilation introducer is then released and passed over the first dilator tube to allow it to progress to the bone, allowing removal of the first dilator tube. This is repeated for the remaining, progressively wider telescoping dilator tubes, to progressively expand the patient's soft tissue down to the entry point on the bone tissue to be treated, and leaving an outer dilator tube port in place. A depth gauge is then used to verify that the appropriate depth has been reached. A pre-drill is advanced to the desired location, which is then also verified by fluoroscopy. A cortex drill is advanced until its positive stop engages, and the distal tip of a tap is driven into the bone until it reaches the appropriate depth, which is then also verified by fluoroscopy. A bone fixation device is then driven into the bone until it reaches the appropriate depth, which is then also verified by fluoroscopy. The bone fixation device is compressed to achieve appropriate stabilization, which is then also verified by fluoroscopy. Once compression of the bone fixation device has been achieved, the pull pin is removed, the guide wire is removed, and the remaining outer dilator tube port is removed.

In a further aspect of the invention, one or more of the dilation tubes may be fitted with light transmitting means, such as optional fibers and the like to illuminate the opening at the distal end of the dilation device to assist in visualizing the area where the procedure is being performed. Similarly, the dilation tubes can be fitted with imaging equipment to allow the surgeon to better observe the procedure being performed.

Other features and advantages of the present invention will become more apparent from the following detailed description of the preferred embodiments in conjunction with the accompanying drawings, which illustrate, by way of example, the operation of the invention.

### BRIEF DESCRIPTION OF THE DRAWINGS

FIG. 1 is a plan view of a first embodiment of a dilation introducer in a locked configuration, according to the present invention.

FIG. 2 is a plan view of the dilation introducer of FIG. 1 shown in an unlocked, collapsed configuration.

FIG. 3 is a plan view of the first or inner dilator tube of the dilation introducer of FIG. 1.

FIG. 4 is a plan view of the second or intermediate dilator tube of the dilation introducer of FIG. 1.

FIG. 5 is a plan view of the third or outer dilator tube of the dilation introducer of FIG. 1.

FIG. 6A is a top plan view of the first locking clip of the dilation introducer of FIG. 1.

FIG. 6B is an elevational view of the first locking clip of the dilation introducer of FIG. 1.

FIG. 6C is a bottom plan view of the first locking clip of the dilation introducer of FIG. 1.

FIG. 7A is a top plan view of the second locking clip of the dilation introducer of FIG. 1.

FIG. 7B is an elevational view of the second locking clip of the dilation introducer of FIG. 1.

FIG. 8 is a perspective view of a second embodiment of a dilation introducer in a locked configuration, according to the present invention.

FIG. 9 is a perspective view of the dilation introducer of FIG. 8 shown in an unlocked, collapsed configuration.

FIG. 10 is a perspective view of the first or inner dilator tube of the dilation introducer of FIG. 8.

FIG. 11 is a perspective view of the second or intermediate dilator tube of the dilation introducer of FIG. 8.

FIG. 12 is a plan view of the third or outer dilator tube of the dilation introducer of FIG. 8.

FIG. 13 is a plan view of a third embodiment of a dilation introducer in a locked configuration, according to the present invention.

FIG. 14 is a plan view of the dilation introducer of FIG. 13 shown in an unlocked, collapsed configuration.

FIG. 15 is a plan view of the first or inner dilator tube of the dilation introducer of FIG. 13.

FIG. 16 is a plan view of the second or intermediate dilator tube of the dilation introducer of FIG. 13.

FIG. 17 is a plan view of the third or outer dilator tube of the dilation introducer of FIG. 13.

FIG. 18 is a plan view of the plastic sleeve of the dilation introducer of FIG. 13.

FIG. 19 is a plan view of a fourth embodiment of a dilation introducer in a locked configuration, according to the present invention.

FIG. 20 is a plan view of the dilation introducer of FIG. 19 shown in an unlocked, collapsed configuration.

FIG. 21 is a plan view of the first or inner dilator tube of the dilation introducer of FIG. 19.

FIG. 22 is a plan view of the second or intermediate dilator tube of the dilation introducer of FIG. 19.

FIG. 23 is a plan view of the third or outer dilator tube of the dilation introducer of FIG. 19.

FIG. 24 is a schematic diagram illustrating location of a starting point for insertion of a bone fixation device according to the method of the invention.

FIG. 25 is a schematic diagram of a lateral view illustrating location of a trajectory for insertion of a bone fixation device according to the method of the invention.

FIG. 26 is a schematic diagram of an anterior view illustrating location of a trajectory for insertion of a bone fixation device according to the method of the invention.

FIG. 27 is a plan view of a guide wire for use with the various embodiments of the telescoping dilation introducer of the invention.

FIG. 28 is a plan view of a second guide wire for use with the various embodiments of the telescoping dilation introducer of the invention.

FIG. 29 is a plan view of a third guide wire for use with the various embodiments of the telescoping dilation introducer of the invention.

FIG. 30 is a perspective view of a variation of the outer dilator tube of the embodiment of FIGS. 8-12, with a parallel guide.

FIG. 31 is a perspective view of the parallel guide from FIG. 30.

FIG. 32 is a perspective view of a variation of the outer dilator tube of the embodiment of FIGS. 8-12, with an angled tip and with a parallel guide.

FIG. 33 is a perspective view of the parallel guide with an angled tip from FIG. 32.

### DETAILED DESCRIPTION OF THE PREFERRED EMBODIMENT

Referring to the drawings, which are provided for purposes of illustration and by way of example, the present invention provides for a telescoping dilation introducer for orthopedic surgery, the dilation introducer having a locked assembled configuration for initial placement of the dilation introducer against a patient's bone tissue to be treated, and an unlocked, collapsed configuration dilating the patient's soft tissue down to the bone tissue to be treated to a desired degree of dilation to permit minimally invasive surgical procedures on the patient's bone tissue to be treated.

While the invention will be described with specificity to a spinal fusion procedure, those skilled in the art will recognize that the apparatus and method of the art will recognize that the apparatus and method of the invention can also be advantageously used for procedures in which the dilation introducer can be brought up against other firm or solid structures in the body or introduced into the body to thereby gain the advantages of the invention for other minimally invasive procedures.

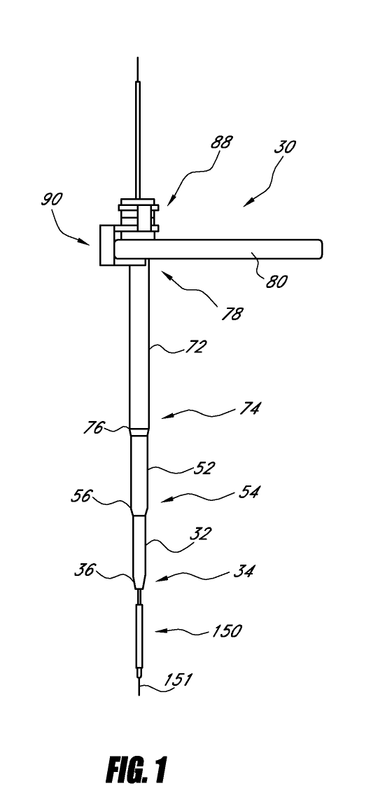

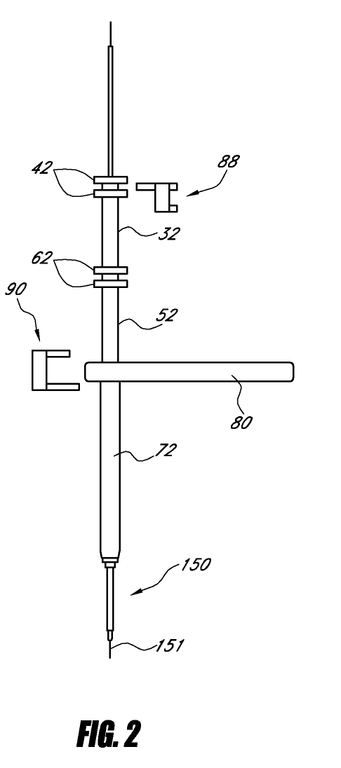

A dilation introducer 30 according to a first preferred embodiment is shown in a locked assembled configuration in FIG. 1, and shown in an unlocked, collapsed configuration in FIG. 2. Referring to FIG. 3, the dilation introducer includes a first or inner dilator tube 32 having a distal end 34 with a tapered tip 36, and a proximal end 38 with a head 40 including a pair of spaced part rings 42. The first dilator tube has an inner lumen 44 with a distal opening 46 and a proximal opening 48.

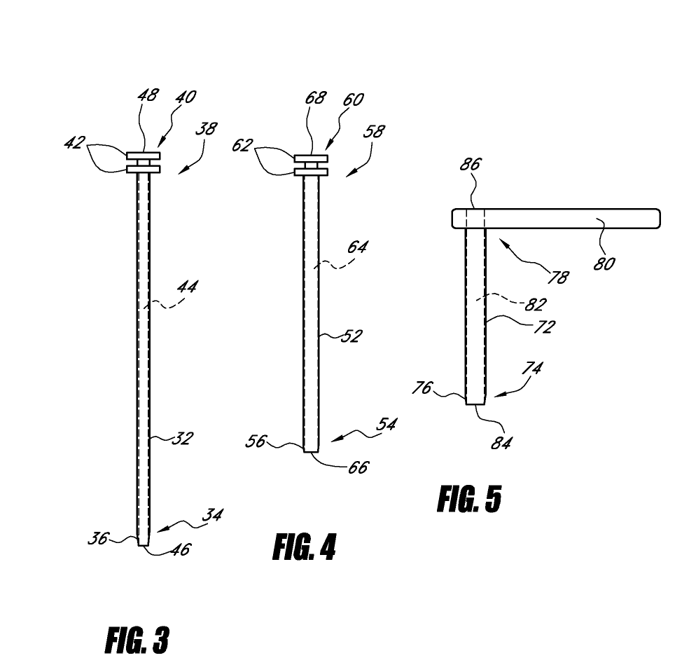

Referring to FIG. 4, the dilation introducer also includes a shorter second or intermediate dilator tube 52 having a distal end 54 with a tapered tip 56, and a proximal end 58 with a head 60 including a pair of spaced apart rings 62. The second dilator tube has an inner lumen 64 with a distal opening 66 and a proximal opening 68.

Referring to FIG. 5, in a presently preferred aspect, the dilation introducer also includes at least one additional dilator tube, such as a still shorter third or outer dilator tube 72 having a distal end 74 with a tapered tip 76, and a proximal end 78 with a handle 80. The third dilator tube has an inner lumen 82 with a distal opening 84 and a proximal opening 86.

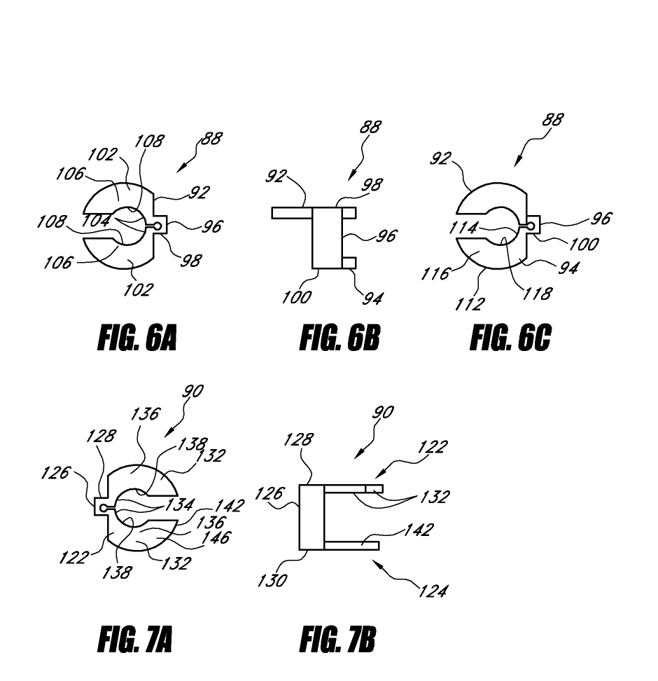

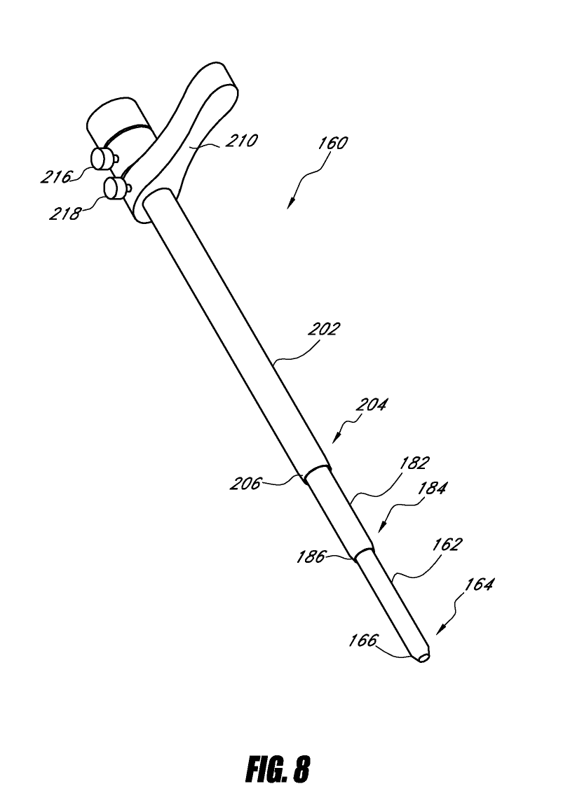

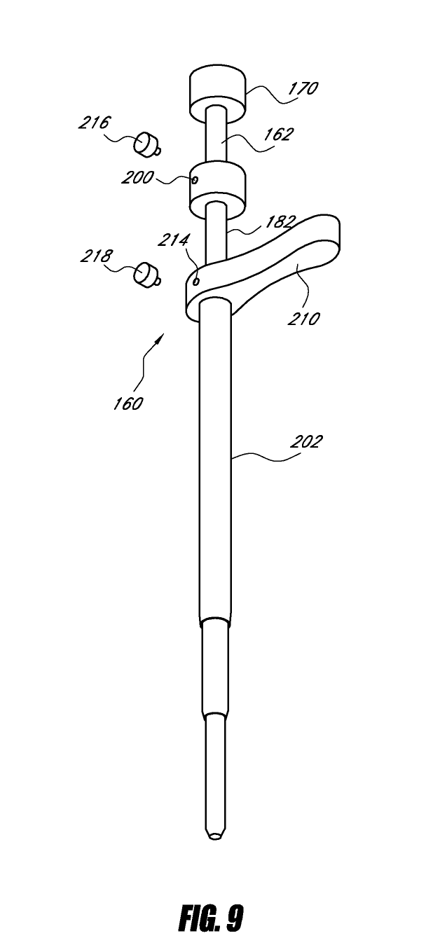

Referring to FIGS. 6A, 6B and 6C, the means for removably connecting the first and second dilator tubes together in a locked configuration includes a first locking clip 88. As is shown in FIGS. 7A and 7B, a means for removably connecting the second and third dilator tubes together in a locked configuration may also be provided, and may include a second locking clip 90. The first locking clip includes a first portion 92 and a second portion 94, and a cross-piece or handle 96 having a first end 98 and a second end 100 connected at right angles between the first and second portions. The first portion includes pair of resilient arms 102 each having a proximal narrow neck portion 104 connected to the cross-piece, and a distal gripping portion 106 extending from the narrow neck portion. The resilient arms have an inner rounded surface 108 adapted to snap over the first dilator tube between the spaced apart rings of the first dilator tube. The second portion currently preferably includes a single arm 112 having a proximal narrow neck portion 114, and a distal gripping portion 116 extending from the narrow neck portion. The gripping portion has an inner rounded surface 118 adapted to fit over the outer surface of the second dilator tube between the spaced apart rings of the second dilator tube, to connect the first and second dilator tubes. Removing the first locking clip allows the second or intermediate dilator tube to slidably telescope over the first inner dilator tube to dilate tissue at the distal end of the dilation introducer.

The second locking clip includes a first portion 122 and a second portion 124, and a cross-piece or handle 126 having a first end 128 and a second end 130 connected between the first portion and the second portion at right angles. The first portion includes a pair of resilient arms 132 each having a proximal narrow neck portion 134 connected to the cross-piece, and a distal gripping portion 136 extending from the narrow neck portion. The pair of resilient arms have an inner rounded surface 138 adapted to snap over the outer surface of the second dilator tube between the spaced apart rings of the second dilator tube. The second portion of the second locking clip includes a pair of resilient arms 142 each having a proximal narrow neck portion (not shown) connected to the cross-piece and a distal gripping portion 146 extending from the narrow neck portion, the pair of resilient arms having an inner rounded surface (not shown) adapted to fit over the outer surface of the third dilator tube to connect the second and third dilator tubes. Removing the second locking clip allows the third or outer dilator tube to slidably telescope over the second inner dilator tube to further dilate tissue at the distal end of the dilation introducer.

As is shown in FIG. 1, a tubular bone drill or tap 150 can be inserted through the inner dilator tube, and the tubular bone drill or tap can be passed or threaded over a guide wire or K wire 151 to contact the surface of the vertebra or bone to be treated, as will be further described below. Once the outer dilator tube has been moved to the distal end of the dilation introducer into position against the vertebra or bone to be treated to fully dilate the soft tissue, the inner dilator tube, the tubular bone drill, and the intermediate dilator tube can be withdrawn and removed to leave the outer dilator tube in place to permit further surgical procedures.

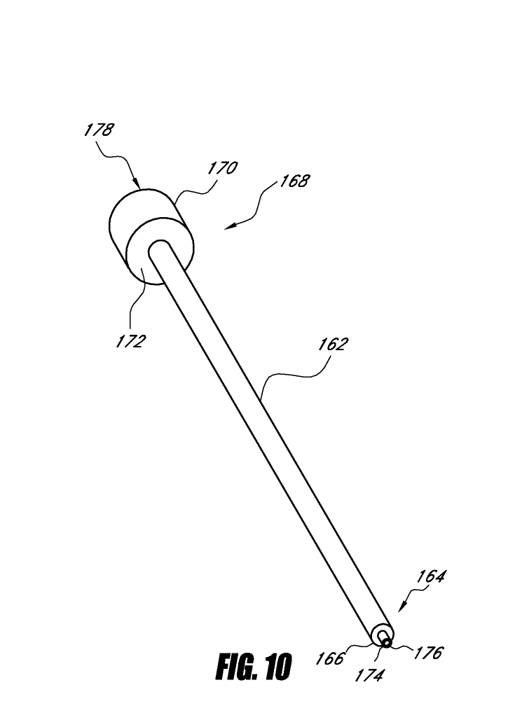

Referring to FIGS. 8-12, the invention provides for a second presently preferred embodiment of a dilation introducer 160 shown in a locked assembled configuration in FIG. 8, and shown in an unlocked, collapsed configuration in FIG. 9. Referring to FIG. 10, the dilation introducer includes a first or inner dilator tube 162 having a distal end 164 with a tapered tip 166, and a proximal end 168 with a cylindrical head 170. The means for removably connecting the first and second dilator tubes together in a locked configuration includes a latching member 172, such as a hook, projecting from the cylindrical head toward the distal end, receiving a locking pin 216, although other latching members, such as a projection with aperture for receiving a locking pin may also be suitable, as will be apparent from the explanation below. The first dilator tube has an inner lumen 174 with a distal opening 176 and a proximal opening 178.

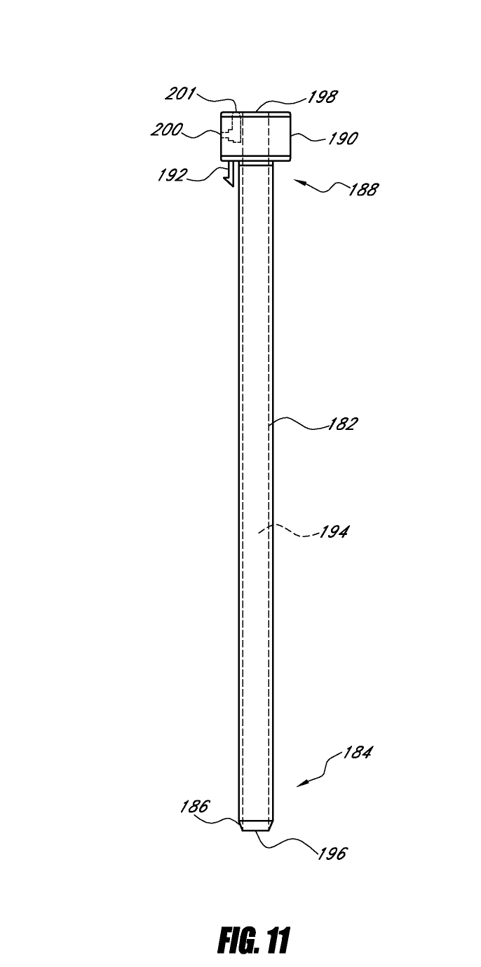

Referring to FIG. 11, the dilation introducer includes a shorter second or intermediate dilator tube 182 having a distal end 184 with a tapered tip 186, and a proximal end 188 having a cylindrical head 190. The means for removably connecting the second and third dilator tubes together in a locked configuration includes a latching member 192, such as a hook, projecting from the cylindrical head toward the distal end, receiving a locking pin 218, although other latching members, such as a projection with aperture for receiving a locking pin may also be suitable, as noted above. The second dilator tube has an inner lumen 194 with a distal opening 196, and a proximal opening 198. The cylindrical head includes a first radial aperture 200 for receiving the locking pin 216, and a second longitudinal aperture 201 for receiving the distally projecting latching member of the cylindrical head of the first or inner dilator tube.

Referring to FIG. 12, in a preferred aspect, the dilation introducer includes at least one additional dilator tube, such as a still shorter third or outer dilator tube 202 having a distal end 204 with a tapered tip 206, and a proximal end 208 to which a handle 210 is connected at its head end 212. The head end of the handle includes a radial aperture 214 for receiving the locking pin 218, and a longitudinal aperture 215 for receiving the distally projecting latching member of the cylindrical head of the second or intermediate dilator tube. The first locking pin 216 is substantially the same as the second locking pin 218. The third dilator tube has an inner lumen 219 with proximal and distal openings. A tubular bone drill or tap can be inserted through the first or inner dilator tube, and the tubular bone drill or tap can be threaded over a guide wire or K wire to contact the surface of the vertebra or bone to be treated, as described above.

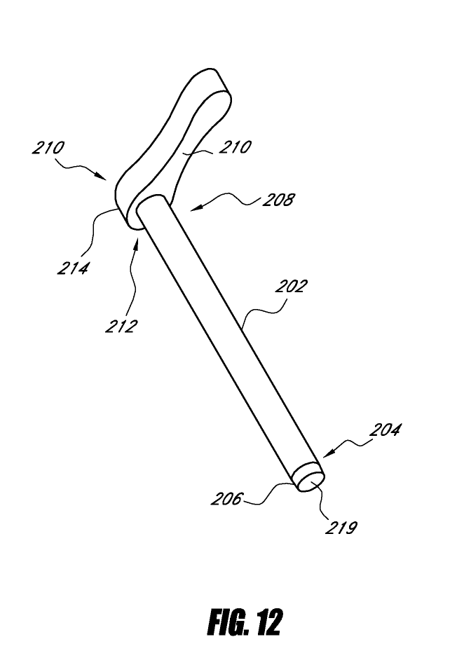

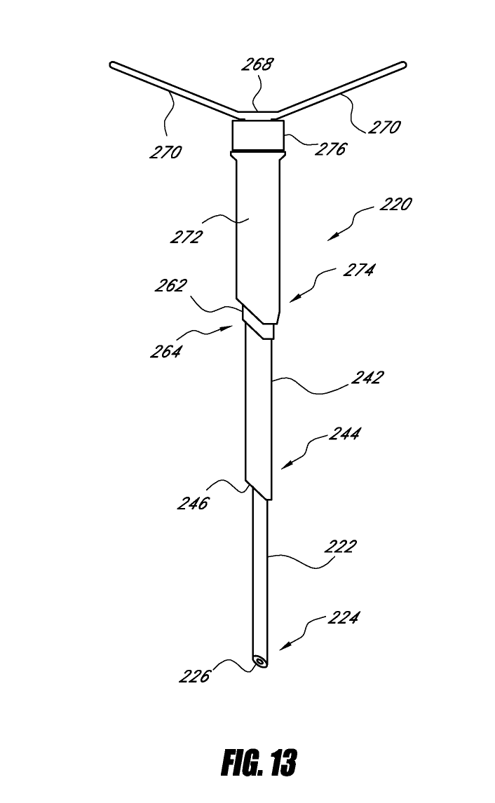

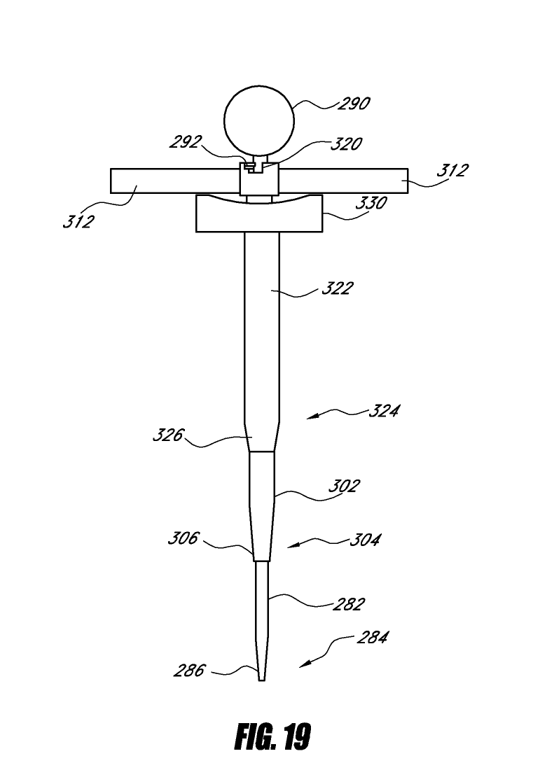

With reference to FIGS. 13-18, the invention provides for a third presently preferred embodiment of a dilation introducer 220, shown in a locked assembled configuration in FIG. 13, and shown in an unlocked, collapsed configuration in FIG. 14. As is illustrated in FIG. 15, the dilation introducer includes a first or inner dilator tube 222 having a distal end 224 with a tapered, beveled tip 226, and a proximal end 228 with a cylindrical head 230. The means for removably connecting the first and second dilator tubes together in a locked configuration includes a pair of opposing bayonet pins 232 extending from the proximal end of the first dilator tube. The first dilator tube has an inner lumen 234 with a distal opening 236 and a proximal opening 238.

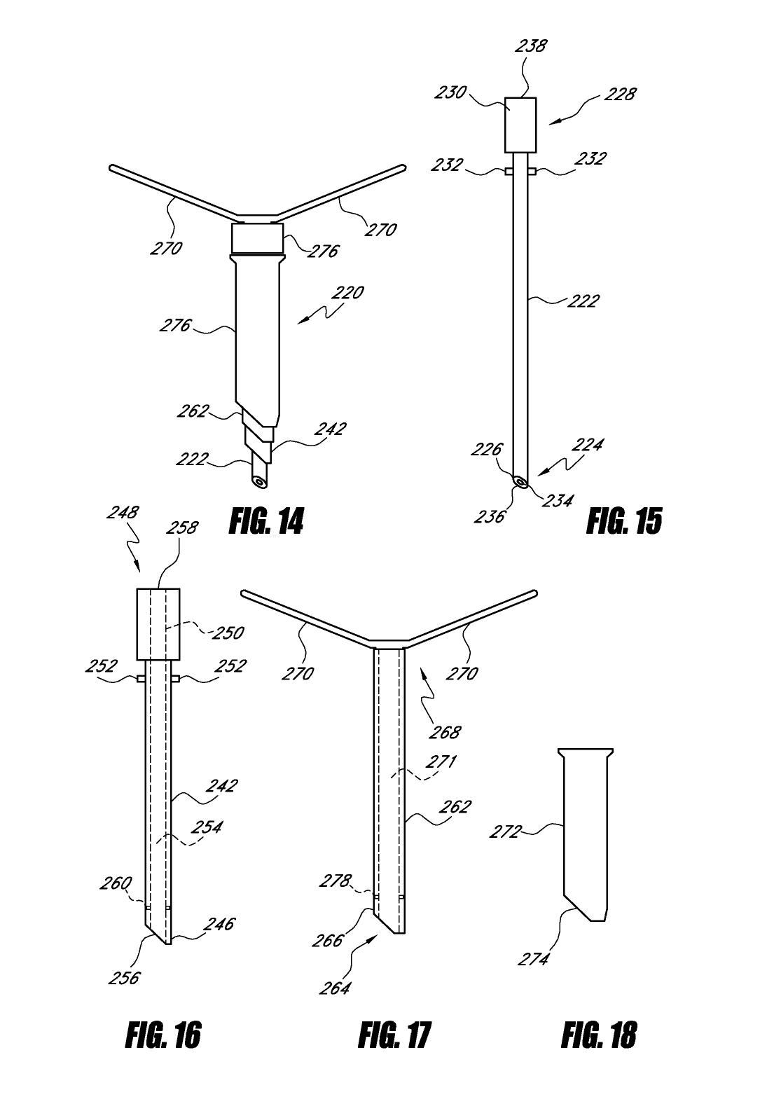

As is shown in FIG. 16, the dilation introducer includes a shorter second or intermediate dilator tube 242 having a distal end 244 with a tapered, beveled tip 246, and a proximal end 248 with a cylindrical head 250. In a preferred aspect, a means for removably connecting the second and third dilator tubes together in a locked configuration includes a pair of opposing bayonet pins 252. The second dilator tube has an inner lumen 254 with a distal opening 256 and a proximal opening 258, and as part of the means for removably connecting the second and third dilator tubes together, interior opposing bayonet slots 260 for receiving the pair of opposing bayonet pins of the first or inner dilator tube.

Referring to FIG. 17, in a preferred aspect, the dilation introducer includes at least one additional dilator tube, such as a still shorter third or outer dilator tube 262 having a distal end 264 with a tapered tip 266, and a proximal end 268 having a pair of opposing handles 270. The third dilator tube has an inner lumen 271, with proximal and distal openings. In another presently preferred aspect, a plastic sleeve 272 is slidably disposed over the shaft of the third or outer dilator tube, and the plastic sleeve preferably has a distal tapered, beveled end 274. A proximal sleeve ring 276 may also be slidably disposed over the shaft of the third or outer dilator tube between the plastic sleeve 272 and the opposing handles.

As is illustrated in FIGS. 13 and 14, in this embodiment the tapered tips of the dilator tubes and plastic sleeve are beveled or angled at a common angle with respect to the longitudinal axis of the dilation introducer, so that the beveled edges of the tapered tips of the dilator tubes and plastic sleeve can be aligned together generally parallel to the surface of the soft tissue to be dilated, so that the bore and dilation passage of the dilation introducer may be aligned at a predetermined desired angle with respect to the soft tissue to be dilated and the bone tissue to be treated.

As part of the means for removably connecting the second and third dilator tubes together, the third dilator tube includes interior opposing bayonet slots 278 for receiving the pair of opposing bayonet pins of the second or intermediate dilator tube. A tubular bone drill or tap can be inserted through the first or inner dilator tube, and the tubular bone drill or tap can be threaded over a guide wire or K wire to contact the surface of the vertebra or bone to be treated, as described above.

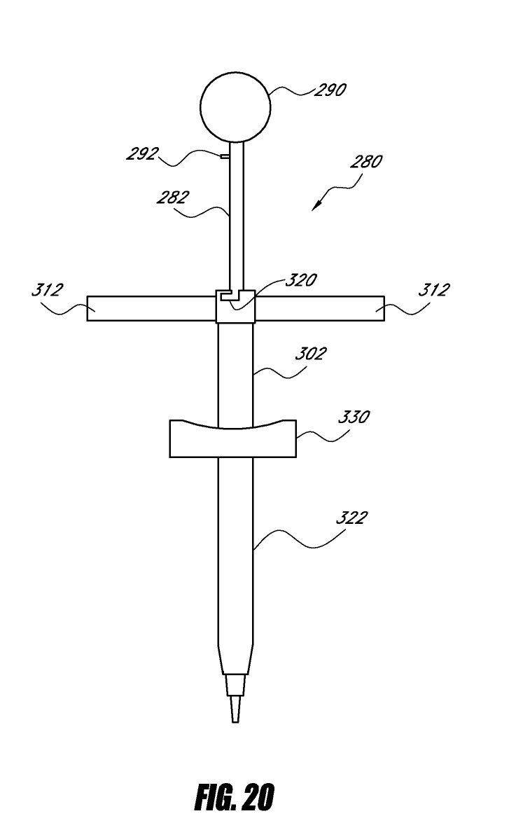

With reference to FIGS. 19-23, the invention provides for a fourth embodiment of a dilation introducer 280 shown in a locked assembled configuration in FIG. 19, and shown in an unlocked, collapsed configuration in FIG. 20. Referring to FIG. 21, the dilation introducer includes a first or inner dilator tube 282 having a distal end 284 with a tapered tip 286, and a proximal end 288 having a generally spherical handle or head 290. As part of a means for removably connecting first and second dilator tubes together in a locked configuration, the proximal end of the first dilator tube near the handle includes a bayonet pin 292. The first dilator tube has an inner lumen 294 with a distal opening 296, and a proximal opening 298.

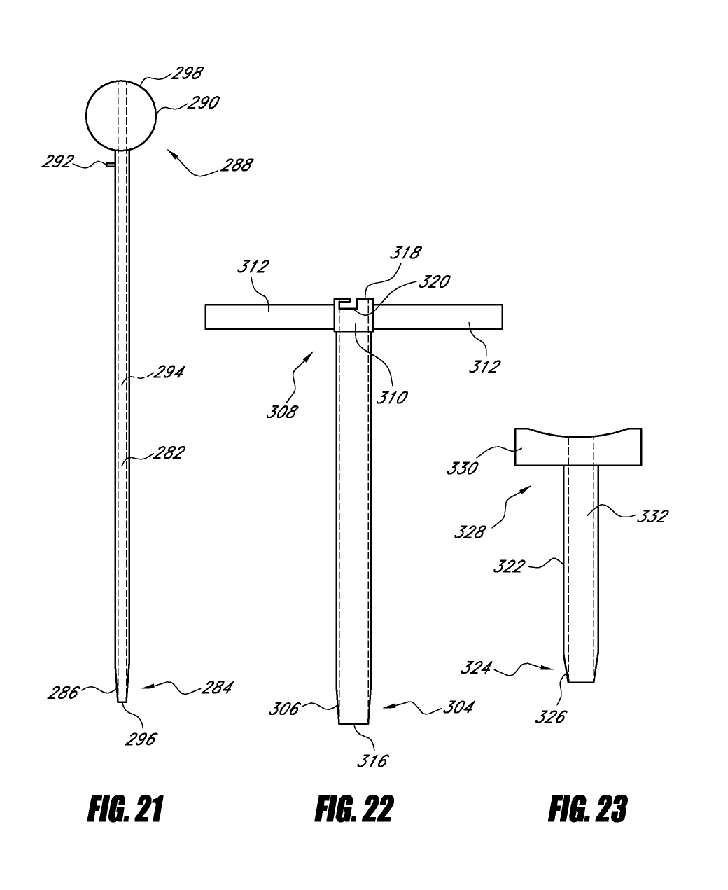

Referring to FIG. 22, the dilation introducer includes a shorter second or intermediate dilator tube 302 having a distal end 304 with a tapered tip 306, and a proximal end 308 having a generally cylindrical head 310 and a pair of opposing handles 312. The second dilator tube has an inner lumen 314 with a distal opening 316 and a proximal opening 318. As part of the means for removably connecting first and second dilator tubes together in a locked configuration, the proximal end of the second dilator tube includes a bayonet slot 320 formed in the cylindrical head for receiving the bayonet pin of the first or inner dilator tube.

Referring to FIG. 23, in a preferred aspect, the dilation introducer includes at least one additional dilator tube, such as a still shorter third or outer dilator tube 322, currently preferably formed of plastic, having a distal end 324 with a tapered tip 326, and a proximal end 328 with a generally cylindrical head end or handle 330. The third dilator tube has an inner lumen 332, with proximal and distal openings. A tubular bone drill or tap can be inserted through the first or inner dilator tube, and the tubular bone drill or tap can be threaded over a guide wire or K wire to contact the surface of the vertebra or bone to be treated, as described above.

### Facet Screw Surgical Technique:

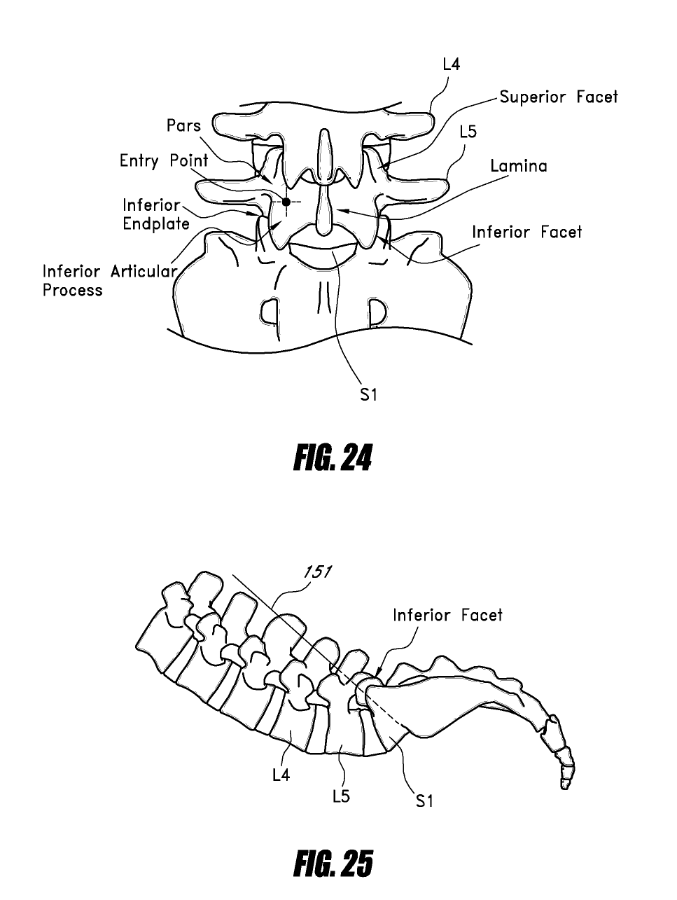

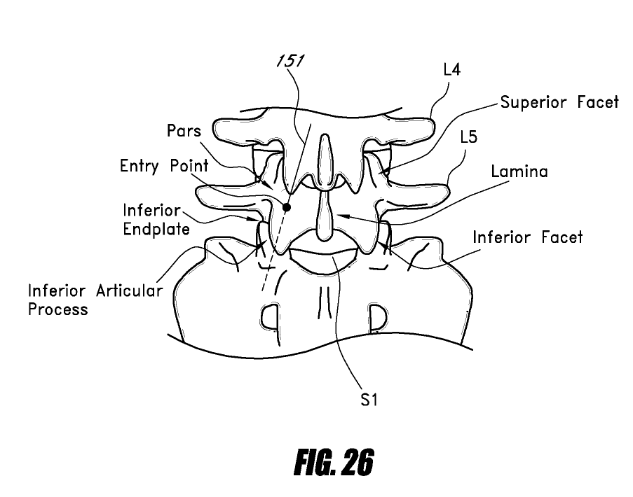

Referring to FIGS. 24-26, a surgical method for spinal fusion utilizing the dilation introducer apparatus and a bone fixation device such as a bone fixation device available under the trade name BONE-LOK from Triage Medical, Inc. of Irvine, Calif., is described. Alternatively, other types of bone screws or fixation devices may also be suitable. The method of the invention involves dilating a patient's soft tissue down to bone tissue to be treated in orthopedic surgery, and necessarily entails an incision and fluoroscopy to locate an entry point on the bone tissue to be treated.

An entry point is located on the bone tissue to be treated, and the tip of a guide wire or K-wire 151 is placed at the entry point on the bone tissue to be treated shown in FIG. 25, and driven into the soft tissue of the patient to the target point of the inferior articular facet. A vertical midline incision to a desired depth, such as approximately 17 mm, is made in the skin and fascia of the patient, using the entry point as the middle of the incision. A first dilator tube of the dilation introducer is then passed over the guide wire until the tip of the dilation introducer reaches the target point of the bone. The guide wire is then driven into the facet joint and into the pedicle of the patient, with verification of the trajectory and depth by fluoroscopy. The second dilator tube of the dilation introducer is then released and passed over the first dilator tube to allow it to progress to the bone, allowing removal of the first dilator tube. This is repeated for the remaining, progressively wider telescoping dilator tubes, to progressively expand the patient's soft tissue down to the entry point on the bone tissue to be treated, and leaving an outer dilator tube port in place. A depth gauge is then used to verify that the appropriate depth has been reached. A pre-drill is advanced to the desired location, which is then also verified by fluoroscopy. A cortex drill is advanced until its positive stop engages, and the distal tip of a tap is driven into the bone until it reaches the appropriate depth, which is then also verified by fluoroscopy. The drill can be connected through an AO style quick connect, or a Jacobs chuck, as long as they are fully cannulated, to a ratcheting handle which is also preferably cannulated. A bone fixation device is then driven into the bone until it reaches the appropriate depth, which is then also verified by fluoroscopy. The bone fixation device is compressed to achieve appropriate stabilization, which is then also verified by fluoroscopy. Once compression of the bone fixation device has been achieved, the pull pin is removed, the guide wire is removed, and the remaining outer dilator tube port is removed, and the incision can be closed normally.

Referring to FIG. 27, in one presently preferred embodiment, a guide wire or K wire 340 for use with the telescoping dilation introducer of the invention includes a proximal enlarged head or stop portion 342, and a relatively narrow elongated body portion 344. The elongated body portion is preferably formed with a proximal section 346 having a relatively larger diameter to provide relatively greater strength, rigidity and torquability for manipulation of the guide wire, a relatively narrower diameter main section 348 to provide the main section with relatively greater flexibility than the proximal section, and a blunt frustoconical distal end 350.

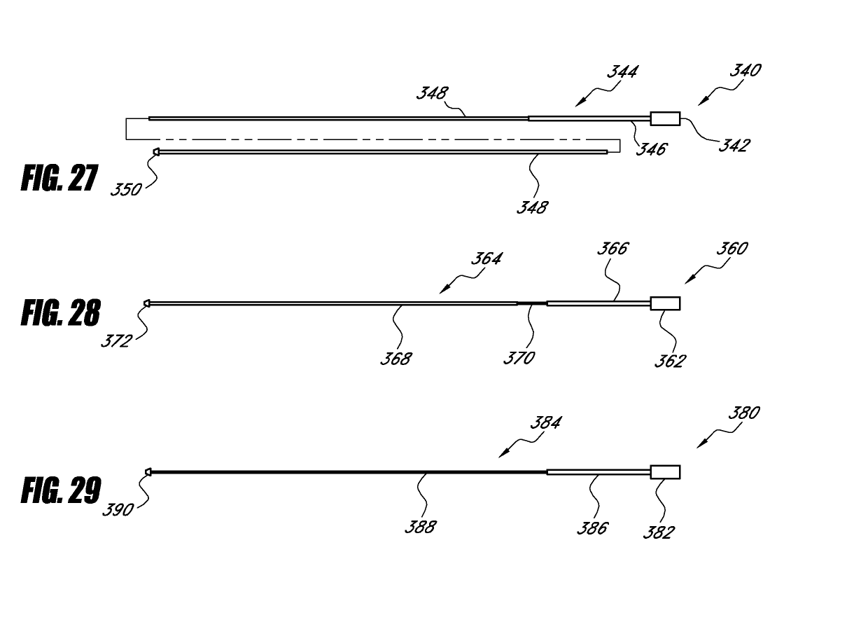

With reference to FIG. 28, in a second presently preferred embodiment, a guide wire or K wire 360 for use with the telescoping dilation introducer of the invention includes a proximal enlarged head or stop portion 362, and a relatively narrow elongated body portion 364. The elongated body portion is preferably formed with a proximal section 366 having a relatively larger diameter to provide relatively greater strength, rigidity and torquability for manipulation of the guide wire, a relatively narrower diameter main section 368 to provide the main section with relatively greater flexibility than the proximal section, a relatively narrower diameter middle section 370 to provide the guide wire with enhanced flexibility between the proximal section and the main section, and a blunt frustoconical distal end 372.

In a third presently preferred embodiment illustrated in FIG. 29, a guide wire or K wire 380 for use with the telescoping dilation introducer of the invention includes a proximal enlarged head or stop portion 382, and a relatively narrow elongated body portion 384. The elongated body portion is preferably formed with a proximal section 386 having a relatively larger diameter to provide relatively greater strength, rigidity and torquability for manipulation of the guide wire, a relatively narrower diameter main section 388 to provide the main section with relatively greater flexibility than the proximal section, and a pointed distal end 390.

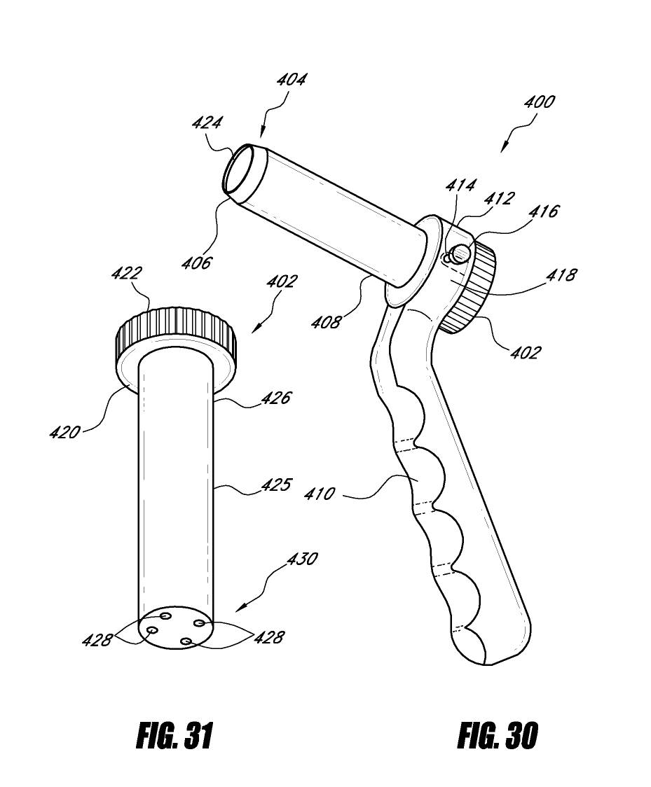

As is shown in FIG. 30, in one presently preferred variation of the at least one additional or outer dilator tube, such as in the embodiment of FIGS. 8-12 for example, the outer dilator tube 400 includes a parallel guide insert 402, shown in FIG. 31. The outer dilator tube has a distal end 404 with a tapered tip 406, and a proximal portion 408 to which a handle 410 is connected at the extreme proximal or head end 412 of the outer dilator tube. The head end of the outer dilator tube includes a radial aperture 414 for receiving the locking pin 416, and a longitudinal aperture 418 for receiving a distally projecting latching member 420 of the cylindrical head 422 of the parallel guide insert. The outer dilator tube has an inner bore 424 with proximal and distal openings.

The parallel guide insert includes a main cylindrical shaft 425 connected at a proximal end 426 to the cylindrical head of the parallel guide insert. The parallel guide insert includes a plurality of longitudinal bores 428 extending the length of the parallel guide insert from the distal end 430, with distal openings visible in FIG. 31, to proximal openings (not shown) in the cylindrical head of the parallel guide insert. The insertion of the distally projecting latching member of the cylindrical head of the parallel guide insert in the longitudinal aperture of the head end of the handle of the outer dilator tube insures that the parallel guide insert remains in a fixed position in the outer dilator tube when the parallel guide insert is secured with the locking pin. A single guide wire or K wire or other device may be passed through one or more of the bores of the parallel guide insert, or multiple guide wires or K wires or other devices may be passed through a plurality of the bores simultaneously, as desired.

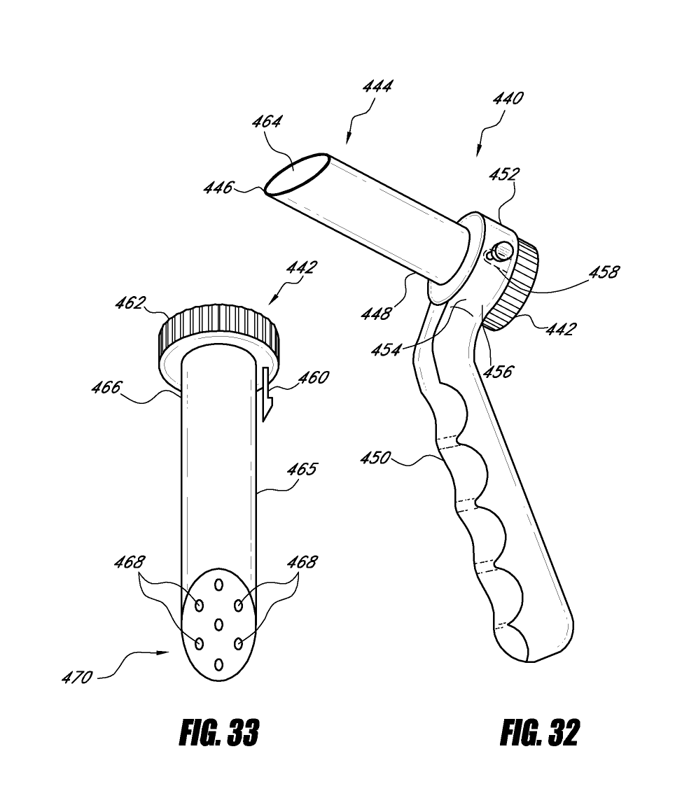

Referring to FIG. 32, in another presently preferred variation of the at least one additional or outer dilator tube, such as in the embodiment of FIGS. 13-18 for example, the outer dilator tube 440 includes a parallel guide insert 442, shown in FIG. 33. The outer dilator tube has a distal end 444 with an angled tip 446, and a proximal end 448 to which a handle 450 is connected at the extreme proximal or head end 452 of the outer dilator tube. The head end of the outer dilator tube includes a radial aperture 454 for receiving the locking pin 456, and a longitudinal aperture 458 for receiving a distally projecting latching member 460 of the cylindrical head 462 of the parallel guide insert. The outer dilator tube has an inner bore 464 with proximal and distal openings.

The parallel guide insert includes a main cylindrical shaft 465 connected at a proximal end 466 to the cylindrical head of the parallel guide insert. The parallel guide insert includes a plurality of longitudinal bores 468 extending the length of the parallel guide insert from the angled distal end 470, with distal openings visible in FIG. 33, to proximal openings (not shown) in the cylindrical head of the parallel guide insert. The insertion of the distally projecting latching member of the cylindrical head of the parallel guide insert in the longitudinal aperture of the head end of the handle of the outer dilator tube insures that the parallel guide insert remains in a fixed position in the outer dilator tube when the parallel guide insert is secured with the locking pin. The angled tips of the outer dilator tube and the parallel guide insert are beveled or angled at a common angle with respect to the longitudinal axis of the dilation introducer, so that the angled tips of the outer dilator tube and the parallel guide insert can be aligned together generally parallel to the surface of the soft tissue to be dilated, with the bore and dilation passage of the dilation introducer aligned at a predetermined desired angle with respect to the soft tissue to be dilated and the bone tissue to be treated. A single guide wire or K wire or other device may be passed through one or more of the bores of the parallel guide insert, or multiple guide wires or K wires or other devices may be passed through a plurality of the bores simultaneously, as desired.

In the foregoing embodiments, the components of the dilation introducer may be formed from plastic, stainless steel, or similar materials or combinations thereof, that can be readily sterilized and packaged ready for use, after which the dilation introducer may be disposed of or resterilized for subsequent use, as desired. The dilator tubes may be radioluscent, with radiopaque markers located on the tips of one or more of the dilator tubes. The tip of the first dilator may also be scored, grooved, or otherwise be provided with a rough surface, to prevent migration. The dilation introducer may also have curved or otherwise non-linear dilator tubes, and the dilation introducer may also have a non-cylindrical shape, such as an oval shape, for example, to allow the dilation introducer to be inserted around objects or a patient's organs.

It should also be appreciated that one or more devices can be inserted through the same dilation introducer, and that the dilation introducer can be repositioned within the same incision for fixation of multiple devices. In addition, fiber optic devices may be inserted through or integrated with the dilation introducer for visual inspection of the target area. While particular locking features have been described for the different embodiments of the dilation introducer, any combination of locking features or alternate locking features may be utilized. The outer dilator tube may not be locked, and a handle on the outer dilator tube may simply be used as a stop. It should also be appreciated that while the invention has been described as being used in the context of orthopedic surgery, and more particularly for implantation of bone fixation devices, the dilation introducer of the invention can also be useful in dilation of soft tissue for percutaneous, minimally invasive surgical procedures such as nephrostomy, neurosurgery, heart valve repair or replacement, gastrointestinal surgery such as for gall bladder or gall stone surgery, hernia removal, transjugular intrahepatic portal-systemic shunt (TIPS) procedures for treatment of the liver, and the like.

It will be apparent from the foregoing that, while particular forms of the invention have been illustrated and described, various modifications can be made without departing from the spirit and scope of the invention. Accordingly, it is not intended that the invention be limited, except as by the appended claims.

What is claimed is:

1. A method of producing a percutaneous telescopic tissue dilation system, the method comprising:
inserting a first dilation tube into a lumen of a second dilation tube; and
engaging a locking assembly with the first and second dilation tubes to maintain at least one of the first dilation tube and the second dilation tube in a locked configuration.

2. The method of claim 1, further comprising inserting both the first dilation tube and the second dilation tube into a lumen of a third dilation tube.

3. The method of claim 2, wherein the third dilation tube comprises a handle extending from the third dilation tube.

4. A method of percutaneously dilating tissue of a patient, the method comprising:
placing a percutaneous telescopic tissue dilation system against a tissue region of a patient, the percutaneous telescopic tissue dilation system comprising a first dilation tube located in a lumen of a second dilation tube and a locking assembly engageable with at least one of the first dilation tube and the second dilation tube;
advancing the first dilation tube into the patient towards a target structure to provide dilation of the tissue around the first dilation tube;
unlocking the second dilation tube to release the second dilation tube from a locked configuration; and
advancing the second dilation tube into the patient towards the target structure to provide greater dilation of the tissue compared to the dilation of the tissue around the first dilation tube.

5. The method of claim 4, wherein the tissue dilation system comprises at least one additional dilation tube having a lumen larger than the second dilation tube, and the method further comprises advancing the at least one additional dilation tube into the patient towards the target structure.

6. The method of claim 5, wherein the at least one additional dilation tube is in an unlocked configuration prior to advancing the at least one additional dilation tube into the patient.

7. The method of claim 5, wherein the at least one additional dilation tube is in a locked configuration with a locking assembly having a different structure than the locking assembly engaging the first dilation tube and the second dilation tube.

8. The method of claim 5, wherein the method further comprises unlocking the at least one additional dilation tube from a locked configuration before advancing the at least one additional dilation tube.

9. The method of claim 4, further comprising inserting a guide wire insert into one of the dilation tubes.

10. The method of claim 9, wherein the guide wire insert comprises a plurality of bores providing access for at least one guide wire to the target structure.

11. The method of claim 9, wherein the guide wire insert has a beveled distal end surface, and the method further comprises placing the beveled distal end surface against a bone surface so that the beveled distal end surface is flush with the bone surface.

12. The method of claim 9, wherein the beveled distal end surface is a concave surface.

13. The method of claim 4, further comprising drilling bone contacted by the tissue dilation system.

14. The method of claim 4, further comprising illuminating the distal end region of one of the dilation tubes.

15. The method of claim 4, further comprising imaging an area in proximity to the distal end of the tissue dilation system.

16. The method of claim 4, further comprising removing the first dilation tube and the second dilation tube from the patient.

17. A method for dilating a patient's soft tissue down to bone tissue to be treated in orthopedic surgery, the method comprising:
locating an entry point on the bone tissue to be treated;
placing a tip of a guide wire down to the entry point on the bone tissue to be treated;
driving the guide wire into the patient's soft tissue to a target point of the bone tissue to be treated;
forming a vertical midline incision to a desired depth in the patient's soft tissue, using the entry point as a middle of the incision;
passing a first dilator tube of a dilation introducer over the guide wire until the first dilator tube reaches the target point of the bone tissue to be treated;
driving the guide wire into the bone tissue to be treated to a desired depth;
passing a second dilator tube of the dilation introducer over the first dilator tube to the bone tissue to be treated, and removing the first dilator tube, leaving the second dilator tube remaining in position in the patient's soft tissue against the bone tissue to be treated;
passing at least one additional dilator tube of the dilation introducer successively over the prior dilator tube remaining in position in the patient's soft tissue against the bone tissue to be treated, to progressively expand the patient's soft tissue down to the entry point on the bone tissue to be treated;
passing a drill over the guide wire to the target point of the bone tissue to be treated, and into the bone tissue to be treated until the drill reaches an appropriate depth;
removing the drill leaving a last remaining dilator tube in place to allow minimally invasive implantation of a bone fixation device over the guide wire in the bone tissue to be treated;
removing the guide wire; and
removing the last remaining dilator tube.
